(19) United States
(12) Patent Application Publication
(10) Pub. No.: US 2010/0248378 A1
(43) Pub. Date: Sep. 30, 2010

(54) METHOD AND MARKER FOR DIAGNOSING DIABETES MELLITUS

(75) Inventor: Harald Mischak, Sehnde (DE)

Correspondence Address: DARDI & HERBERT, PLLC, Moore Lake Plaza, Suite 205, 1250 East Moore Lake Drive, Fridley, MN, 55432, US

(73) Assignee: Mosaiques Diagnostics and Therapeutics AG

(21) Appl. No.: 12/738,775

(22) PCT Filed: Oct. 20, 2008
(86) PCT No.: PCT/EP08/64136

§ 371 Date: Jun. 4, 2010

(30) Foreign Application Priority Data
Oct. 19, 2007 (EP) 07118843.7

(51) Int. Cl. G01N 33/68; G01N 27/26; G06F 19/00

(52) U.S. Cl. 436/86; 204/451; 702/19

(57) ABSTRACT

A process for the diagnosis of diabetes mellitus comprising the step of determining the presence or absence or amplitude of at least three polypeptide markers in a urine sample, the polypeptide markers being selected from the markers characterized by the values for the molecular masses and migration times according to Table 1.

The present invention relates to the diagnosis of diabetes mellitus by the non-invasive analysis of endogenous proteins and peptides.

The number of patients suffering from diabetes mellitus has strongly increased in recent years. This disease represents one of the greatest problems to the health systems.

Diabetes mellitus can be readily controlled clinically in an early phase, and therefore, early diagnosis is very important. Early diagnosis and a therapy that is precisely adapted to this specific disease can reduce the risk of patients becoming afflicted with further associated diseases and becoming blind, for example, or becoming dialysis-dependent. In addition, a well-aimed therapy also reduces the high risk of getting cardiovascular diseases in such patients.

Currently, the diagnosis is based on measurements of the blood glucose level and on the oral glucose tolerance test (OGTT).

Urine analyses are a further approach for the diagnosis of diabetes mellitus.

However, only glucose is measured in the urine today. The diagnostic value of such analyses is limited because of a lack of sufficient sensitivity and selectivity. A possible approach is the examination of proteins and peptides in the urine.

Various attempts have been made to characterize proteins in the urine.

V. Thongboonkerd et al., Kidney International, Vol. 63 (2001), pages 1461-1469, describe two-dimensional polyacrylamide gel electrophoresis (2D-PAGE) in connection with matrix-assisted laser desorption ionization-time-of-flight mass spectrometry (MALDI-TOF) followed by mass fingerprinting for the examination of urine samples. All in all, 67 forms of 47 individual proteins were identified.

C. S. Spahr et al., Proteomics Vol. 1 (2001), pages 93-107, cleaved proteins from urine samples using trypsin and could establish 751 peptides from 124 proteins by means of liquid chromatography and tandem mass spectrometry.

This study involved only healthy subjects. The studies did not deal with the question of whether the detection of changes in the composition of polypeptides in the urine is useful for the diagnosis of diabetes mellitus.

H. Mischak et al. in Clinical Science 107 (2004), pages 485-495, describe proteoma analysis for the evaluation of diabetic kidney lesions. A small number of peptides that occur more often or at a lesser frequency as the disease progresses have been identified.

Due to the small number of samples, a sufficient specificity for the diagnosis cannot be achieved in these works. Therefore, there is still a need for a rapid and simple method for the diagnosis of diabetes mellitus.

Therefore, it is the object of the present invention to provide processes and means for the diagnosis of diabetes mellitus.

This object is achieved by a process for the diagnosis of diabetes mellitus comprising the step of determining the presence or absence or amplitude of at least three polypeptide markers in a urine sample, the polypeptide markers being selected from the markers characterized in Table 1 by values for the molecular masses and migration times.

Using the markers according to the invention, it is possible to achieve a specificity of at least 60%, preferably at least 70%, more preferably at least 80%, even more preferably at least 90% and most preferably at least 95% for diabetes mellitus.

Using the markers according to the invention, it is possible to achieve a sensitivity of at least 60%, preferably at least 70%, more preferably at least 80%, even more preferably at least 90% and most preferably at least 95% for diabetes mellitus.

The evaluation of the measured polypeptides can be effected by means of the presence or absence or amplitude of the markers, taking the following limits as shown in Table 2 into account.

“Control” means healthy subjects and patients with various diseases who are not afflicted with diabetes mellitus.

In the Tables, “MEAN” means the arithmetic mean, i.e., the sum of all values divided by the number of values.

“MEDIAN” is the middle value of a list, i.e., the smallest value for which at least half the values in the list are not greater. For an odd number of values, the median is the middle value of a list sorted in an increasing order. For an even number of values, the median is the sum of the two middle values obtained upon sorting divided by two.

AUC is the so-called “area under the curve”, which is examined within the scope of ROC (receiver-operator characteristic) analysis and which is a measure of the quality of the individual parameter (biomarker), based on the cases examined. Thus, the sensitivity on the ordinate is plotted against 1-specificity on the abscissa in the diagram. Specificity is defined as the number of actually negative samples divided by the sum of the numbers of the actually negative and false positive samples. A specificity of 1 means that a test recognizes all healthy persons as being healthy, i.e., no healthy subject is identified as being ill. This says nothing about how reliably the test recognizes sick patients. Sensitivity is defined as the number of actually positive samples divided by the sum of the numbers of the actually positive and false negative samples. A sensitivity of 1 means that the test recognizes all sick persons. This says nothing about how reliably the test recognizes healthy patients. Thus, an AUC value of 1 means that all samples have been assigned correctly (specificity and sensitivity of 1), an AUC value of 0.5 means that the samples have been assigned with guesswork probability and the parameter thus has no significance.

The method of “Bonferroni” is employed to correct multivariate statistic analyses and yields a corrected p value. The p value states the probability that the differences found are due to random variations. The smaller the p value, the higher is the significance.

Preferably, markers are employed whose AUC value is greater than 0.6, preferably greater than 0.7. Preferably, markers are employed whose Bonferroni corrected p value is smaller than 10⁻⁴, preferably smaller than 10⁻⁵, more preferably smaller than 10⁻⁶.

The migration time is determined by capillary electrophoresis (CE), for example, as set forth in the Example under item 2. In this Example, a glass capillary of 90 cm in length and with an inner diameter (ID) of 50 μm and an outer diameter (OD) of 360 μm is operated at an applied voltage of 30 kV. As the mobile solvent, 30% methanol, 0.5% formic acid in water is used, for example.

It is known that the CE migration times may vary. Nevertheless, the order in which the polypeptide markers are eluted is typically the same under the stated conditions for each CE system employed. In order to balance any differences in the migration time that may nevertheless occur, the system can be normalized using standards for which the migration times are exactly known. These standards may be, for example, the polypeptides stated in the Examples (see the Example, item 3).

The characterization of the polypeptides shown in the Tables was determined by means of capillary electrophoresis-mass spectrometry (CE-MS), a method which has been described in detail, for example, by Neuhoff et al. (Rapid communications in mass spectrometry, 2004, Vol. 20, pages 149-156). The variation of the molecular masses between individual measurements or between different mass spectrometers is relatively small when the calibration is exact, typically within a range of ±0.01% or ±0.005%.

The polypeptide markers according to the invention are proteins or peptides or degradation products of proteins or peptides. They may be chemically modified, for example, by posttranslational modifications, such as glycosylation, phosphorylation, alkylation or disulfide bridges, or by other reactions, for example, within the scope of degradation. In addition, the polypeptide markers may also be chemically altered, for example, oxidized, in the course of the purification of the samples.

Proceeding from the parameters that determine the polypeptide markers (molecular weight and migration time), it is possible to identify the sequence of the corresponding polypeptides by methods known in the prior art.

The polypeptides according to the invention are used to diagnose diabetes mellitus.

“Diagnosis” means the process of knowledge gaining by assigning symptoms or phenomena to a disease or injury. In the present case, the presence or absence of particular polypeptide markers is also used for differential diagnosis. The presence or absence of a polypeptide marker can be measured by any method known in the prior art. Methods which may be used are exemplified below.

A polypeptide marker is considered present if its measured value is at least as high as its threshold value. If the measured value is lower, then the polypeptide marker is considered absent. The threshold value can be determined either by the sensitivity of the measuring method (detection limit) or defined from experience.

In the context of the present invention, the threshold value is considered to be exceeded preferably if the measured value of the sample for a certain molecular mass is at least twice as high as that of a blank sample (for example, only buffer or solvent).

The polypeptide marker or markers is/are used in such a way that its/their presence or absence is measured, wherein the presence or absence is indicative of diabetes mellitus. Thus, there are polypeptide markers which are typically present in patients with diabetes mellitus, but do not or less frequently occur in subjects with no diabetes mellitus. Further, there are polypeptide markers which are present in subjects with diabetes mellitus, but do not or less frequently occur in subjects with no diabetes mellitus.

In addition or also alternatively to the frequency markers (determination of presence or absence), amplitude markers may also be used for diagnosis. Amplitude markers are used in such a way that the presence or absence is not critical, but the height of the signal (the amplitude) is decisive if the signal is present in both groups. In the Tables, the mean amplitudes of the corresponding signals (characterized by mass and migration time) averaged over all samples measured to are stated. To achieve comparability between differently concentrated samples or different measuring methods, two normalization methods are possible. In the first approach, all peptide signals of a sample are normalized to a total amplitude of 1 million counts. Therefore, the respective mean amplitudes of the individual markers are stated as parts per million (ppm).

In addition, it is possible to define further amplitude markers by an alternative normalization method: In this case, all peptide signals of one sample are scaled with a common normalization factor. Thus, a linear regression is formed between the peptide amplitudes of the individual samples and the reference values of all known polypeptides. The slope of the regression line just corresponds to the relative concentration and is used as a normalization factor for this sample.

All groups employed consist of at least 100 individual patient or control samples in order to obtain a reliable mean amplitude. The decision for a diagnosis is made as a function of how high the amplitude of the respective polypeptide markers in the patient sample is in comparison with the mean amplitudes in the control group or the “diabetes mellitus” group. If the value is in the vicinity of the mean amplitude of the “diabetes mellitus” group, the existence of diabetes mellitus is to be considered, and if it rather corresponds to the mean amplitudes of the control group, the non-existence of diabetes mellitus is to be considered. The distance from the mean amplitude can be interpreted as a probability of the sample's belonging to a certain group.

Alternatively, the distance between the measured value and the mean amplitude may be considered a probability of the sample's belonging to a certain group.

A frequency marker is a variant of an amplitude marker in which the amplitude is low in some samples. It is possible to convert such frequency markers to amplitude markers by including the corresponding samples in which the marker is not found into the calculation of the amplitude with a very small amplitude, on the order of the detection limit.

The subject from which the sample in which the presence or absence of one or more polypeptide markers is determined is derived may be any subject which is capable of suffering from diabetes mellitus. Preferably, the subject is a mammal, and most preferably, it is a human.

In a preferred embodiment of the invention, not just three polypeptide markers, but a combination of more polypeptide markers is used to enable a differential diagnosis. The exact diabetes mellitus is concluded from their presence or absence. By comparing a plurality of polypeptide markers, a bias in the overall result due to a few individual deviations from the typical presence probability in the individual can be reduced or avoided.

The sample in which the presence or absence of the peptide marker or markers according to the invention is measured may be any sample which is obtained from the body of the subject. The sample is a sample which has a polypeptide composition suitable for providing information about the state of the subject. For example, it may be blood, urine, a synovial fluid, a tissue fluid, a body secretion, sweat, cerebrospinal fluid, lymph, intestinal, gastric or pancreatic juice, bile, lacrimal fluid, a tissue sample, sperm, vaginal fluid or a feces sample. Preferably, it is a liquid sample.

In a preferred embodiment, the sample is a urine sample.

Urine samples can be taken as preferred in the prior art. Preferably, a midstream urine sample is used in the context of the present invention. For example, the urine sample may be taken by means of a catheter or also by means of a urination apparatus as described in WO 01/74275.

The presence or absence of a polypeptide marker in the sample may be determined by any method known in the prior art that is suitable for measuring polypeptide markers. Such methods are known to the skilled person. In principle, the presence or absence of a polypeptide marker can be determined by direct to methods, such as mass spectrometry, or indirect methods, for example, by means of ligands.

If required or desirable, the sample from the subject, for example, the urine sample, may be pretreated by any suitable means and, for example, purified or separated before the presence or absence of the polypeptide marker or markers is measured. The treatment may comprise, for example, purification, separation, dilution or concentration. The methods may be, for example, centrifugation, filtration, ultrafiltration, dialysis, precipitation or chromatographic methods, such as affinity separation or separation by means of ion-exchange chromatography, or electrophoretic separation. Particular examples thereof are gel electrophoresis, two-dimensional polyacrylamide gel electrophoresis (2D-PAGE), capillary electrophoresis, metal affinity chromatography, immobilized metal affinity chromatography (IMAC), lectin-based affinity chromatography, liquid chromatography, highperformance liquid chromatography (HPLC), normal and reverse-phase HPLC, cation-exchange chromatography and selective binding to surfaces. All these methods are well known to the skilled person, and the skilled person will be able to select the method as a function of the sample employed and the method for determining the presence or absence of the polypeptide marker or markers.

In one embodiment of the invention, the sample, before being measured is separated by capillary electrophoresis, purified by ultracentrifugation and/or divided by ultrafiltration into fractions which contain polypeptide markers of a particular molecular size.

Preferably, a mass-spectrometric method is used to determine the presence or absence of a polypeptide marker, wherein a purification or separation of the sample may be performed upstream from such method. As compared to the currently employed methods, mass-spectrometric analysis has the advantage that the concentration of many (>100) polypeptides of a sample can be determined by a single analysis. Any type of mass spectrometer may be employed. By means of mass spectrometry, it is possible to measure 10 fmol of a polypeptide marker, i.e., 0.1 ng of a 10 kD protein, as a matter of routine with a measuring accuracy of about ±0.01% in a complex mixture. In mass spectrometers, an ion-forming unit is coupled with a suitable analytic device. For example, electrospray-ionization (ESI) interfaces are mostly used to measure ions in liquid samples, whereas MALDI (matrix-assisted laser desorption/ionization) technique is used for measuring ions from a sample crystallized in a matrix. To analyze the ions formed, quadrupoles, ion traps or time-of-flight (TOF) analyzers may be used, for example.

In electrospray ionization (ESI), the molecules present in solution are atomized, inter alia, under the influence of high voltage (e.g., 1-8 kV), which forms charged droplets that become smaller from the evaporation of the solvent. Finally, so-called Coulomb explosions result in the formation of free ions, which can then be analyzed and detected.

In the analysis of the ions by means of TOF, a particular acceleration voltage is applied which confers an equal amount of kinetic energy to the ions. Thereafter, the time that the respective ions take to travel a particular drifting distance through the flying tube is measured very accurately. Since with equal amounts of kinetic energy, the velocity of the ions depends on their mass, the latter can thus be determined. TOF analyzers have a very high scanning speed and therefore reach a good resolution.

Preferred methods for the determination of the presence or absence of polypeptide markers include gas-phase ion spectrometry, such as laser desorption/ionization mass spectrometry, MALDI-TOF MS, SELDI-TOF MS (surface-enhanced laser desorption/ionization), LC MS (liquid chromatography/mass spectrometry), 2D-PAGE/MS and capillary electrophoresis-mass spectrometry (CE-MS). All the methods mentioned are known to the skilled person.

A particularly preferred method is CE-MS, in which capillary electrophoresis is coupled with mass spectrometry. This method has been described in some detail, for example, in the German Patent Application DE 10021737, in Kaiser et al. (I Chromatogr A, 2003, Vol. 1013: 157-171, and Electrophoresis, 2004, 25: 2044-2055) and in Wittke et al. (J. Chromatogr. A, 2003, 1013: 173-181). The CE-MS technology allows to determine the presence of some hundreds of polypeptide markers of a sample simultaneously within a short time and in a small volume with high sensitivity. After a sample has been measured, a pattern of the measured polypeptide markers is prepared, and this pattern can be compared with reference patterns of sick or healthy subjects. In most cases, it is sufficient to use a limited number of polypeptide markers for the diagnosis of UAS. A CE-MS method which includes CE coupled on-line to an ESI-TOF MS is further preferred.

For CE-MS, the use of volatile solvents is preferred, and it is best to work under essentially salt-free conditions. Examples of suitable solvents include acetonitrile, methanol and the like. The solvents can be diluted with water or an acid (e.g., 0.1% to 1% formic acid) in order to protonate the analyte, preferably the polypeptides.

By means of capillary electrophoresis, it is possible to separate molecules by their charge and size. Neutral particles will migrate at the speed of the electroosmotic flow upon application of a current, while cations are accelerated towards the cathode, and anions are delayed. The advantage of capillaries in electrophoresis resides in the favorable ratio of surface to volume, which enables a good dissipation of the Joule heat generated during the current flow. This in turn allows high voltages (usually up to 30 kV) to be applied and thus a high separating performance and short times of analysis.

In capillary electrophoresis, silica glass capillaries having inner diameters of typically from 50 to 75 μm are usually employed. The lengths employed are 30-100 cm. In addition, the capillaries are usually made of plastic-coated silica glass. The capillaries may be either untreated, i.e., expose their hydrophilic groups on the interior surface, or coated on the interior surface. A hydrophobic coating may be used to improve the resolution. In addition to the voltage, a pressure may also be applied, which typically is within a range of from 0 to 1 psi. The pressure may also be applied only during the separation or altered meanwhile.

In a preferred method for measuring polypeptide markers, the markers of the sample are separated by capillary electrophoresis, then directly ionized and transferred on-line into a coupled mass spectrometer for detection.

In the method according to the invention, it is advantageous to use several polypeptide markers for the diagnosis.

The use of at least 5, 6, 8 or 10 markers is preferred.

In one embodiment, from 20 to 50 markers are used. More preferably, all the markers described here are used.

Preferably, at least three markers are used whose mean AUC is greater than 0.65 and whose mean Bonferroni corrected p value is smaller than 10⁻⁵.

Preferably, three or more markers are selected in such a way that the average of the quotients of AUC divided by Bonferroni corrected p values are greater than 1000, preferably greater than 10,000, more preferably greater than 100,000.

In order to determine the probability of the existence of a disease when several markers are used, statistic methods known to the skilled person may be used. For example, the Random Forests method described by Weissinger et al. (Kidney Int., 2004, 65: 2426-2434) may be used by using a computer program such as S-Plus, or the support vector machines as described in the same publication.

Table 1 states the mass in daltons and the CE time in minutes.

Table 2 states the frequency of the corresponding markers in normal controls (non-diabetic subjects) and diabetes patients, averages (mean and median) of the amplitudes and the Bonferroni corrected p values.

Table 3 states the protein sequence and the assignment to known proteins for part of the markers.

### EXAMPLE

### 1. Sample Preparation

For detecting the polypeptide markers for the diagnosis, urine was employed. Urine was collected from healthy donors and patients not suffering from diabetes mellitus (control group) as well as from patients suffering from diabetes mellitus.

For the subsequent CE-MS measurement, the proteins which are also contained in the urine of patients in an elevated concentration, such as albumin and immunoglobulins, had to be separated off by ultrafiltration. Thus, 700 μl of urine was collected and admixed with 700 μl of filtration buffer (2 M urea, 10 mM ammonia, 0.02% SDS). This 1.4 ml of sample volume was ultrafiltrated (20 kDa, Sartorius, Göttingen, Germany). The ultrafiltration was performed at 3000 rpm in a centrifuge until 1.1 ml of ultrafiltrate was obtained.

The 1.1 ml of filtrate obtained was then applied to a PD 10 column (Amersham Bioscience, Uppsala, Sweden) and desalted against 2.5 ml of 0.01% NH₄OH, and lyophilized. For the CE-MS measurement, the polypeptides were then resuspended with 20 μl of water (HPLC grade, Merck).

### 2. CE-MS Measurement

The CE-MS measurements were performed with a Beckman Coulter capillary electrophoresis system (P/ACE MDQ System; Beckman Coulter Inc., Fullerton, Calif., USA) and a Bruker ESI-TOF mass spectrometer (micro-TOF MS, Bruker Daltonik, Bremen, Germany).

The CE capillaries were supplied by Beckman Coulter and had an ID/OD of 50/360 μm and a length of 90 cm. The mobile phase for the CE separation consisted of 20% acetonitrile and 0.25% formic acid in water. For the “sheath flow” on the MS, 30% isopropanol with 0.5% formic acid was used, here at a flow rate of 2 μl/min. The coupling of CE and MS was realized by a CE-ESI-MS Sprayer Kit (Agilent Technologies, Waldbronn, Germany).

For injecting the sample, a pressure of from 1 to a maximum of 6 psi was applied, and the duration of the injection was 99 seconds. With these parameters, about 150 nl of the sample was injected into the capillary, which corresponds to about 10% of the capillary volume. A stacking technique was used to concentrate the sample in the capillary. Thus, before the sample was injected, a 1 M NH₃ solution was injected for 7 seconds (at 1 psi), and after the sample was injected, a 2 M formic acid solution was injected for 5 seconds. When the separation voltage (30 kV) was applied, the analytes were automatically concentrated between these solutions.

The subsequent CE separation was performed with a pressure method: 40 minutes at 0 psi, then 0.1 psi for 2 min, 0.2 psi for 2 min, 0.3 psi for 2 min, 0.4 psi for 2 min, and finally 0.5 psi for 32 min. The total duration of a separation run was thus 80 minutes.

In order to obtain as good a signal intensity as possible on the side of the MS, the nebulizer gas was turned to the lowest possible value. The voltage applied to the spray needle for generating the electrospray was 3700-4100 V. The remaining settings at the mass spectrometer were optimized for peptide detection according to the manufacturer's instructions. The spectra were recorded over a mass range of m/z 400 to m/z 3000 and accumulated every 3 seconds.

### 3. Standards for the CE Measurement

For checking and standardizing the CE measurement, the following proteins or polypeptides which are characterized by the stated CE migration times under the chosen conditions were employed:

| | |
|---|---|
| Protein/polypeptide | Migration time |

| | |
|---|---|
| Aprotinin (SIGMA, Taufkirchen, DE, Cat. No. A1153) | 19.3 min |
| Ribonuclease, SIGMA, Taufkirchen, DE, Cat. No. R4875 | 19.55 min |
| Lysozyme, SIGMA, Taufkirchen, DE, Cat. No. L76511 | 9.28 min |
| “REV”, Sequence: REVQSKIGYGRQIIS | 20.95 min |
| “ELM”, Sequence: ELMTGELPYSHINNRDQIIFMVGR | 23.49 min |
| “KINCON”, Sequence: TGSLPYSHIGSRDQIIFMVGR | 22.62 min |
| “GIVLY” Sequence: GIVLYELMTGELPYSHIN | 32.2 min |

The proteins/polypeptides were employed at a concentration of 10 pmol/μl each in water. “REV”, “ELM, “KINCON” and “GIVLY” are synthetic peptides.

The molecular masses of the peptides and the m/z ratios of the individual charge states visible in MS are stated in the following Table:

| | H (mono) | | | | | | |
|---|---|---|---|---|---|---|---|
| | 1.0079 | 1.0079 | 1.0079 | 1.0079 | 1.0079 | 1.0079 | 1.0079 |
| | Aprotinin | Ribonuclease | Lysozyme | REV | KINCON | ELM | GIVLY |
| m/z | Mono Mass | Mono Mass | Mono Mass | Mono Mass | Mono Mass | Mono Mass | Mono Mass |
| 0 | 6513.09 | 13681.32 | 14303.88 | 1732.96 | 2333.19 | 2832.41 | 2048.03 |
| 1 | 6514.0979 | 13682.328 | 14304.888 | 1733.9679 | 2334.1979 | 2833.4179 | 2049.0379 |
| 2 | 3257.5529 | 6841.6679 | 7152.9479 | 867.4879 | 1167.6029 | 1417.2129 | 1025.0229 |
| 3 | 2172.0379 | 4561.4479 | 4768.9679 | 578.6612 | 778.7379 | 945.1446 | 683.6846 |
| 4 | 1629.2804 | 3421.3379 | 3576.9779 | 434.2479 | 584.3054 | 709.1104 | 513.0154 |
| 5 | 1303.6259 | 2737.2719 | 2861.7839 | 347.5999 | 467.6459 | 567.4899 | 410.6139 |
| 6 | 1086.5229 | 2281.2279 | 2384.9879 | 289.8346 | 389.8729 | 473.0762 | 342.3462 |
| 7 | 931.4494 | 1955.4822 | 2044.4193 | 248.5736 | 334.3208 | 405.6379 | 293.5836 |
| 8 | 815.1442 | 1711.1729 | 1788.9929 | 217.6279 | 292.6567 | 355.0592 | 257.0117 |
| 9 | 724.6846 | 1521.1546 | 1590.3279 | 193.559 | 260.2512 | 315.7201 | 228.5668 |
| 10 | 652.3169 | 1369.1399 | 1431.3959 | 174.3039 | 234.3269 | 284.2489 | 205.8109 |
| 11 | 593.107 | 1244.7643 | 1301.3606 | 158.5497 | 213.1161 | 258.4997 | 187.1924 |
| 12 | 543.7654 | 1141.1179 | 1192.9979 | 145.4212 | 195.4404 | 237.0421 | 171.6771 |
| 13 | 502.0148 | 1053.4171 | 1101.3063 | 134.3125 | 180.4841 | 218.8856 | 158.5486 |

In principle, it is known to the skilled person that slight variations of the migration times may occur in separations by capillary electrophoresis. However, under the conditions described, the order of migration will not change. For the skilled person who knows the stated masses and CE times, it is possible without difficulty to assign their own measurements to the polypeptide markers according to the invention. For example, they may proceed as follows: At first, they select one of the polypeptides found in their measurement (peptide 1) and try to find one or more identical masses within a time slot of the stated CE time (for example, ±5 min). If only one identical mass is found within this interval, the assignment is completed. If several matching masses are found, a decision about the assignment is still to be made. Thus, another peptide (peptide 2) from the measurement is selected, and it is tried to identify an appropriate polypeptide marker, again taking a corresponding time slot into account.

Again, if several markers can be found with a corresponding mass, the most probable assignment is that in which there is a substantially linear relationship between the shift for peptide 1 and that for peptide 2.

Depending on the complexity of the assignment problem, it suggests itself to the skilled person to optionally use further proteins from their sample for assignment, for example, ten proteins. Typically, the migration times are either extended or shortened by particular absolute values, or compressions or expansions of the whole course occur. However, comigrating peptides will also comigrate under such conditions.

In addition, the skilled person can make use of the migration patterns described by Zuerbig et al. in Electrophoresis 27 (2006), pp. 2111-2125. If they plot their measurement in the form of m/z versus migration time by means of a simple diagram (e.g., with MS Excel), the line patterns described also become visible. Now, a simple assignment of the individual polypeptides is possible by counting the lines.

Other approaches of assignment are also possible. Basically, the skilled person could also use the peptides mentioned above as internal standards for assigning their CE measurements.

TABLE 1

| No. | Mass (Da) | CE T (min) |
|---|---|---|
| 1 | 858.39 | 23.24 |
| 2 | 884.32 | 24.85 |
| 3 | 911.43 | 25.88 |
| 4 | 981.59 | 24.8 |
| 5 | 984.46 | 24.92 |
| 6 | 1013.37 | 25.17 |
| 7 | 1018.46 | 24.54 |
| 8 | 1040.47 | 25.05 |
| 9 | 1050.48 | 26.92 |
| 10 | 1058.48 | 24.89 |
| 11 | 1070.49 | 36.49 |
| 12 | 1071.49 | 21.43 |
| 13 | 1080.48 | 27.77 |
| 14 | 1080.5 | 25.69 |
| 15 | 1096.48 | 26.08 |
| 16 | 1100.5 | 37.04 |
| 17 | 1114.49 | 25.55 |
| 18 | 1128.49 | 25.65 |
| 19 | 1141.52 | 24.51 |
| 20 | 1141.54 | 37.33 |
| 21 | 1143.52 | 36.97 |
| 22 | 1153.31 | 35.61 |
| 23 | 1157.54 | 37.44 |
| 24 | 1162.54 | 20.11 |
| 25 | 1173.53 | 37.49 |
| 26 | 1180.52 | 35.7 |
| 27 | 1182.55 | 28.27 |
| 28 | 1186.53 | 22.39 |
| 29 | 1191.52 | 36.18 |
| 30 | 1210.39 | 36.48 |
| 31 | 1211.54 | 25.82 |
| 32 | 1216.54 | 24.24 |
| 33 | 1217.53 | 35.78 |
| 34 | 1260.56 | 21.83 |
| 35 | 1262.46 | 38.23 |
| 36 | 1263.54 | 22.73 |
| 37 | 1265.59 | 27.09 |
| 38 | 1270.55 | 29.38 |
| 39 | 1276.4 | 35.92 |
| 40 | 1281.58 | 27.09 |
| 41 | 1297.58 | 27.36 |
| 42 | 1299.58 | 22.38 |
| 43 | 1312.55 | 29.77 |
| 44 | 1312.62 | 22.45 |
| 45 | 1324.59 | 28.7 |
| 46 | 1326.55 | 29.2 |
| 47 | 1337.62 | 38.2 |
| 48 | 1351.64 | 38.76 |
| 49 | 1353.66 | 25.63 |
| 50 | 1367.64 | 38.88 |
| 51 | 1378.61 | 28.82 |
| 52 | 1383.64 | 38.94 |
| 53 | 1396.62 | 26.67 |
| 54 | 1405.64 | 20.14 |
| 55 | 1407.66 | 37.23 |
| 56 | 1408.66 | 39.13 |
| 57 | 1422.68 | 28.14 |
| 58 | 1424.66 | 39.3 |
| 59 | 1438.66 | 30.2 |
| 60 | 1438.67 | 27.88 |
| 61 | 1439.66 | 29.82 |
| 62 | 1440.66 | 39.28 |
| 63 | 1442.63 | 27.63 |
| 64 | 1458.63 | 27.94 |
| 65 | 1470.68 | 21.08 |
| 66 | 1482.67 | 22.47 |
| 67 | 1485.67 | 23.77 |
| 68 | 1486.68 | 21.15 |
| 69 | 1491.74 | 39.83 |
| 70 | 1494.66 | 30.4 |
| 71 | 1496.68 | 30.38 |
| 72 | 1507.74 | 40.02 |
| 73 | 1508.68 | 29.33 |
| 74 | 1510.68 | 20.17 |
| 75 | 1513.44 | 36.79 |
| 76 | 1521.69 | 30.53 |
| 77 | 1523.74 | 40.66 |
| 78 | 1523.84 | 29.75 |
| 79 | 1525.48 | 37.17 |
| 80 | 1526.69 | 23.92 |
| 81 | 1549.7 | 39.49 |
| 82 | 1551.66 | 22.29 |
| 83 | 1552.5 | 37.22 |
| 84 | 1608.68 | 22.35 |
| 85 | 1608.73 | 30.93 |
| 86 | 1612.76 | 23.38 |
| 87 | 1624.55 | 37.73 |
| 88 | 1638.73 | 20.23 |
| 89 | 1640.58 | 23.24 |
| 90 | 1640.68 | 28.04 |
| 91 | 1654.78 | 23.13 |
| 92 | 1664.75 | 29.81 |
| 93 | 1669.69 | 21.47 |
| 94 | 1692.8 | 30.89 |
| 95 | 1697.74 | 30.88 |
| 96 | 1698.57 | 37.73 |
| 97 | 1716.77 | 28 |
| 98 | 1725.59 | 38.32 |
| 99 | 1732.77 | 28.17 |
| 100 | 1749.81 | 30.61 |
| 101 | 1750.78 | 23.83 |
| 102 | 1764.68 | 19.91 |
| 103 | 1765.81 | 31 |
| 104 | 1769.71 | 28.14 |
| 105 | 1793.88 | 32.37 |
| 106 | 1798.72 | 36.95 |
| 107 | 1809.88 | 32.3 |
| 108 | 1817.69 | 20.23 |
| 109 | 1818.83 | 30.95 |
| 110 | 1822.73 | 30.87 |
| 111 | 1823.99 | 24.4 |
| 112 | 1835.71 | 19.91 |
| 113 | 1837.8 | 30.56 |
| 114 | 1840.84 | 41.18 |
| 115 | 1847.89 | 43.67 |
| 116 | 1860.83 | 21.4 |
| 117 | 1880.9 | 43.91 |
| 118 | 1885.65 | 38.82 |
| 119 | 1892.86 | 24.33 |
| 120 | 1915.91 | 31.3 |
| 121 | 1916.77 | 20.32 |
| 122 | 1934.79 | 19.94 |
| 123 | 1942.84 | 30.96 |
| 124 | 1945 | 33.71 |
| 125 | 1962.88 | 31.81 |
| 126 | 1963.88 | 31.74 |
| 127 | 1996.79 | 20.98 |
| 128 | 2034.99 | 40.19 |
| 129 | 2039.13 | 21.78 |
| 130 | 2055.94 | 25.44 |
| 131 | 2058.94 | 23.15 |
| 132 | 2067.82 | 20.62 |
| 133 | 2070.92 | 25.4 |
| 134 | 2076.95 | 21.78 |
| 135 | 2080.94 | 20.2 |
| 136 | 2128.98 | 26.97 |
| 137 | 2132.91 | 25.83 |
| 138 | 2137.94 | 21.79 |
| 139 | 2156.97 | 22.22 |
| 140 | 2168.97 | 32.91 |
| 141 | 2189 | 26.89 |
| 142 | 2191.99 | 22.39 |
| 143 | 2194.97 | 20.17 |
| 144 | 2216.03 | 33.83 |
| 145 | 2226.99 | 26.28 |
| 146 | 2235.04 | 34.17 |
| 147 | 2248.99 | 25.99 |
| 148 | 2249.04 | 20.53 |
| 149 | 2257.87 | 35.93 |
| 150 | 2276.02 | 27.23 |
| 151 | 2277.01 | 27.23 |
| 152 | 2280.94 | 36.22 |
| 153 | 2289.04 | 33.59 |
| 154 | 2292.02 | 27.28 |
| 155 | 2308.02 | 27.34 |
| 156 | 2319.07 | 33.82 |
| 157 | 2323.04 | 22.36 |
| 158 | 2339 | 34.01 |
| 159 | 2367.06 | 27.63 |
| 160 | 2377.1 | 20.8 |
| 161 | 2385.05 | 33.95 |
| 162 | 2421 | 34.86 |
| 163 | 2446.09 | 28.37 |
| 164 | 2471.16 | 34.77 |
| 165 | 2483.12 | 27.57 |
| 166 | 2485.13 | 34.41 |
| 167 | 2525.2 | 27.74 |
| 168 | 2544.13 | 28.26 |
| 169 | 2559.18 | 19.41 |
| 170 | 2570.19 | 42.56 |
| 171 | 2583.15 | 23.68 |
| 172 | 2584.23 | 35.18 |
| 173 | 2587.2 | 21.1 |
| 174 | 2596.23 | 34.9 |
| 175 | 2612.21 | 34.9 |
| 176 | 2639.29 | 21.42 |
| 177 | 2644.22 | 21.15 |
| 178 | 2654.19 | 23.92 |
| 179 | 2668.25 | 41.97 |
| 180 | 2748.79 | 36.38 |
| 181 | 2751.34 | 29.23 |
| 182 | 2756.27 | 35.24 |
| 183 | 2767.32 | 21.67 |
| 184 | 2802.82 | 36.34 |
| 185 | 2839.35 | 24.2 |
| 186 | 2907.35 | 35.96 |
| 187 | 2912.17 | 25.56 |
| 188 | 2926.3 | 22.22 |
| 189 | 2939.15 | 33.77 |
| 190 | 2946.21 | 34.98 |
| 191 | 2973.45 | 24.37 |
| 192 | 3001.43 | 35.4 |
| 193 | 3002.24 | 23.8 |
| 194 | 3011.39 | 29.75 |
| 195 | 3013.29 | 22.3 |
| 196 | 3035.19 | 42.02 |
| 197 | 3064.32 | 20.57 |
| 198 | 3081.42 | 29.83 |
| 199 | 3091.44 | 28.4 |
| 200 | 3108.45 | 31.28 |
| 201 | 3139.49 | 29.48 |
| 202 | 3149.46 | 31.25 |
| 203 | 3165.46 | 31.32 |
| 204 | 3178.43 | 30.3 |
| 205 | 3193.38 | 22.64 |
| 206 | 3209.41 | 22.67 |
| 207 | 3256.53 | 33.03 |
| 208 | 3281.43 | 36.09 |
| 209 | 3292.54 | 39.42 |
| 210 | 3318.55 | 30.99 |
| 211 | 3333.72 | 23.83 |
| 212 | 3334.54 | 31.02 |
| 213 | 3337.45 | 22.81 |
| 214 | 3350.55 | 31.02 |
| 215 | 3359.58 | 31.9 |
| 216 | 3363.54 | 30.22 |
| 217 | 3385.55 | 25.49 |
| 218 | 3401.6 | 25.47 |
| 219 | 3405.48 | 25.97 |
| 220 | 3426.31 | 27.7 |
| 221 | 3575.75 | 32.36 |
| 222 | 3657.67 | 40.71 |
| 223 | 3696.76 | 26.94 |
| 224 | 3718.72 | 32.48 |
| 225 | 3734.72 | 32.5 |
| 226 | 3831.81 | 28.48 |
| 227 | 3870.81 | 33.49 |
| 228 | 3871.79 | 27.57 |
| 229 | 3943.83 | 33.63 |
| 230 | 4002.62 | 20.66 |
| 231 | 4047.92 | 25.45 |
| 232 | 4078.81 | 33.14 |
| 233 | 4217.98 | 26.05 |
| 234 | 4251.98 | 28.76 |
| 235 | 4289.93 | 28.78 |
| 236 | 4368.9 | 20.21 |
| 237 | 4436.08 | 26.32 |
| 238 | 4467.96 | 29.12 |
| 239 | 4630 | 29.38 |
| 240 | 4863.16 | 26.74 |
| 241 | 8917.25 | 22.55 |

TABLE 2

| | | | Normal control | Normal control mean | Diabetes | | Bonferroni |
|---|---|---|---|---|---|---|---|
| Mass | CE T | AUC | frequency | (median) amp | frequency | Diabetes mean (median) amp | corrected p value |
| 858.39 | 23.24 | 0.6586 | 0.83 | 2.33 (2.34) | 0.63 | 2.28 (2.25) | 0.0435652 |
| 884.32 | 24.85 | 0.6104 | 0.66 | 2.29 (2.28) | 0.81 | 2.47 (2.42) | 0.0263486 |
| 911.43 | 25.88 | 0.6586 | 0.87 | 2.71 (2.72) | 0.72 | 2.51 (2.61) | 0.0027488 |
| 981.59 | 24.8 | 0.6787 | 0.95 | 2.81 (2.89) | 0.7 | 2.66 (2.73) | 5.72E−12 |
| 984.46 | 24.92 | 0.6345 | 0.32 | 1.81 (1.85) | 0.49 | 1.95 (2.03) | 0.0018068 |
| 1013.37 | 25.17 | 0.6847 | 0.93 | 2.94 (2.93) | 0.95 | 3.32 (3.35) | 0.0430425 |
| 1018.46 | 24.54 | 0.6406 | 0.44 | 2.05 (2.09) | 0.59 | 2.32 (2.40) | 0.000435 |
| 1040.47 | 25.05 | 0.6867 | 0.8 | 2.37 (2.41) | 0.51 | 2.32 (2.38) | 0.0001277 |
| 1050.48 | 26.92 | 0.7068 | 0.97 | 2.81 (2.84) | 0.8 | 2.57 (2.62) | 1.471E−08 |
| 1058.48 | 24.89 | 0.7992 | 0.08 | 1.77 (1.68) | 0.53 | 2.93 (3.06) | 4.26E−16 |
| 1070.49 | 36.49 | 0.5904 | 0.52 | 2.24 (2.27) | 0.27 | 2.06 (2.07) | 4.881E−05 |
| 1071.49 | 21.43 | 0.6185 | 0.61 | 2.16 (2.17) | 0.32 | 1.94 (1.94) | 1.807E−06 |
| 1080.48 | 27.77 | 0.6827 | 0.66 | 2.19 (2.22) | 0.29 | 2.10 (2.23) | 7.221E−06 |
| 1080.5 | 25.69 | 0.6606 | 0.62 | 2.21 (2.24) | 0.26 | 2.12 (2.17) | 7.60E−12 |
| 1096.48 | 26.08 | 0.7711 | 0.98 | 3.78 (3.80) | 0.81 | 3.26 (3.49) | 3.02E−11 |
| 1100.5 | 37.04 | 0.6265 | 0.7 | 2.31 (2.36) | 0.46 | 2.13 (2.17) | 0.0004769 |
| 1114.49 | 25.55 | 0.7369 | 0.96 | 3.55 (3.60) | 0.85 | 3.24 (3.41) | 4.12E−06 |
| 1128.49 | 25.65 | 0.7008 | 0.8 | 2.44 (2.47) | 0.5 | 2.20 (2.27) | 6.13E−10 |
| 1141.52 | 24.51 | 0.6365 | 0.66 | 2.27 (2.28) | 0.38 | 2.05 (2.06) | 1.857E−06 |
| 1141.54 | 37.33 | 0.6647 | 0.75 | 2.52 (2.57) | 0.48 | 2.32 (2.26) | 2.385E−07 |
| 1143.52 | 36.97 | 0.6908 | 0.87 | 2.65 (2.70) | 0.64 | 2.39 (2.42) | 1.596E−09 |
| 1153.31 | 35.61 | 0.5683 | 0.67 | 2.55 (2.58) | 0.69 | 2.57 (2.62) | 0.0424232 |
| 1157.54 | 37.44 | 0.7269 | 0.95 | 3.21 (3.27) | 0.82 | 2.88 (2.92) | 7.29E−10 |
| 1162.54 | 20.11 | 0.5944 | 0.61 | 2.34 (2.39) | 0.36 | 2.19 (2.20) | 0.0136225 |
| 1173.53 | 37.49 | 0.6707 | 0.77 | 2.47 (2.52) | 0.51 | 2.17 (2.17) | 1.47E−11 |
| 1180.52 | 35.7 | 0.7088 | 0.79 | 2.78 (2.83) | 0.39 | 2.40 (2.49) | 6.26E−11 |
| 1182.55 | 28.27 | 0.7229 | 0.66 | 2.08 (2.11) | 0.16 | 1.97 (1.96) | 6.62E−16 |
| 1186.53 | 22.39 | 0.739 | 0.87 | 2.87 (2.88) | 0.66 | 2.48 (2.47) | 1.702E−09 |
| 1191.52 | 36.18 | 0.7068 | 0.83 | 2.65 (2.72) | 0.48 | 2.33 (2.35) | 2.08E−11 |
| 1210.39 | 36.48 | 0.6345 | 0.63 | 2.57 (2.58) | 0.33 | 2.48 (2.50) | 0.0131101 |
| 1211.54 | 25.82 | 0.6566 | 0.63 | 2.10 (2.10) | 0.3 | 1.99 (2.00) | 5.189E−07 |
| 1216.54 | 24.24 | 0.7088 | 0.87 | 3.14 (3.15) | 0.79 | 2.77 (2.82) | 0.0381734 |
| 1217.53 | 35.78 | 0.6747 | 0.76 | 3.49 (3.61) | 0.41 | 3.17 (3.29) | 5.698E−10 |
| 1260.56 | 21.83 | 0.6767 | 0.38 | 2.39 (2.38) | 0.56 | 2.67 (2.66) | 0.0302727 |
| 1262.46 | 38.23 | 0.6044 | 0.51 | 2.23 (2.27) | 0.21 | 1.95 (2.01) | 3.471E−10 |
| 1263.54 | 22.73 | 0.6426 | 0.81 | 2.59 (2.60) | 0.63 | 2.57 (2.57) | 0.023065 |
| 1265.59 | 27.09 | 0.7771 | 0.95 | 3.78 (3.80) | 0.67 | 3.47 (3.46) | 1.39E−13 |
| 1270.55 | 29.38 | 0.6687 | 0.66 | 2.27 (2.29) | 0.29 | 2.05 (2.07) | 0.0001114 |
| 1276.4 | 35.92 | 0.5964 | 0.98 | 3.54 (3.57) | 0.97 | 3.41 (3.41) | 0.0023336 |
| 1281.58 | 27.09 | 0.6787 | 0.75 | 2.47 (2.52) | 0.41 | 2.26 (2.31) | 8.713E−08 |
| 1297.58 | 27.36 | 0.745 | 0.86 | 3.17 (3.21) | 0.41 | 3.05 (3.09) | 1.80E−16 |
| 1299.58 | 22.38 | 0.7129 | 0.84 | 2.51 (2.54) | 0.48 | 2.41 (2.47) | 5.19E−10 |
| 1312.55 | 29.77 | 0.5462 | 0.96 | 3.15 (3.20) | 0.95 | 3.08 (3.09) | 2.657E−07 |
| 1312.62 | 22.45 | 0.5803 | 0.74 | 2.71 (2.76) | 0.79 | 2.80 (2.81) | 0.018991 |
| 1324.59 | 28.7 | 0.6124 | 0.45 | 2.50 (2.52) | 0.66 | 2.63 (2.71) | 0.0001963 |
| 1326.55 | 29.2 | 0.6687 | 0.8 | 2.39 (2.42) | 0.49 | 2.16 (2.17) | 7.645E−08 |
| 1337.62 | 38.2 | 0.6767 | 0.65 | 2.34 (2.37) | 0.3 | 1.89 (1.90) | 2.92E−17 |
| 1351.64 | 38.76 | 0.7008 | 0.8 | 2.57 (2.60) | 0.49 | 2.27 (2.28) | 2.01E−12 |
| 1353.66 | 25.63 | 0.7129 | 0.91 | 2.70 (2.72) | 0.79 | 2.40 (2.47) | 6.629E−09 |
| 1367.64 | 38.88 | 0.747 | 0.94 | 2.98 (3.03) | 0.78 | 2.61 (2.66) | 3.73E−13 |
| 1378.61 | 28.82 | 0.7369 | 0.99 | 3.57 (3.57) | 0.97 | 3.33 (3.35) | 4.196E−05 |
| 1383.64 | 38.94 | 0.6225 | 0.54 | 2.27 (2.29) | 0.23 | 1.94 (1.96) | 4.281E−06 |
| 1396.62 | 26.67 | 0.5522 | 0.61 | 2.13 (2.14) | 0.47 | 2.02 (2.03) | 0.0002984 |
| 1405.64 | 20.14 | 0.6406 | 0.59 | 2.44 (2.48) | 0.26 | 2.06 (2.08) | 3.016E−09 |
| 1407.66 | 37.23 | 0.6968 | 0.73 | 2.47 (2.51) | 0.32 | 2.09 (2.11) | 2.03E−14 |
| 1408.66 | 39.13 | 0.6888 | 0.85 | 2.89 (2.94) | 0.66 | 2.58 (2.63) | 2.051E−09 |
| 1422.68 | 28.14 | 0.7088 | 0.83 | 3.47 (3.49) | 0.52 | 3.52 (3.51) | 9.995E−08 |
| 1424.66 | 39.3 | 0.6908 | 0.93 | 3.47 (3.53) | 0.82 | 3.11 (3.21) | 5.458E−09 |
| 1438.66 | 30.2 | 0.5884 | 0.51 | 2.19 (2.23) | 0.26 | 2.14 (2.22) | 7.114E−06 |
| 1438.67 | 27.88 | 0.747 | 0.97 | 3.54 (3.55) | 0.9 | 3.27 (3.41) | 0.0006262 |
| 1439.66 | 29.82 | 0.6365 | 0.56 | 2.22 (2.22) | 0.64 | 2.37 (2.34) | 0.000113 |
| 1440.66 | 39.28 | 0.6647 | 0.8 | 2.76 (2.80) | 0.52 | 2.58 (2.62) | 7.215E−09 |
| 1442.63 | 27.63 | 0.739 | 0.2 | 1.93 (1.97) | 0.53 | 2.48 (2.49) | 4.70E−10 |
| 1458.63 | 27.94 | 0.6727 | 0.38 | 2.29 (2.33) | 0.55 | 2.69 (2.75) | 0.0033535 |
| 1470.68 | 21.08 | 0.6064 | 0.52 | 2.09 (2.06) | 0.24 | 1.84 (1.86) | 1.93E−06 |
| 1482.67 | 22.47 | 0.5723 | 0.56 | 2.67 (2.70) | 0.41 | 2.40 (2.39) | 0.0091356 |
| 1485.67 | 23.77 | 0.745 | 0.98 | 3.11 (3.14) | 0.88 | 2.84 (2.89) | 1.101E−07 |
| 1486.68 | 21.15 | 0.6807 | 0.8 | 2.57 (2.57) | 0.5 | 2.28 (2.29) | 1.71E−10 |
| 1491.74 | 39.83 | 0.7108 | 0.88 | 2.83 (2.87) | 0.65 | 2.49 (2.52) | 3.34E−13 |
| 1494.66 | 30.4 | 0.6586 | 0.86 | 2.31 (2.32) | 0.68 | 2.26 (2.26) | 0.0279139 |
| 1496.68 | 30.38 | 0.6165 | 0.63 | 2.25 (2.24) | 0.37 | 2.07 (2.05) | 0.0017352 |
| 1507.74 | 40.02 | 0.7149 | 0.93 | 3.58 (3.61) | 0.89 | 3.21 (3.23) | 5.548E−08 |
| 1508.68 | 29.33 | 0.7108 | 0.97 | 3.49 (3.52) | 0.88 | 3.27 (3.40) | 0.0005901 |
| 1510.68 | 20.17 | 0.6667 | 0.35 | 2.25 (2.27) | 0.53 | 2.54 (2.58) | 0.0001918 |
| 1513.44 | 36.79 | 0.6225 | 0.59 | 2.41 (2.40) | 0.31 | 2.26 (2.16) | 2.318E−05 |
| 1521.69 | 30.53 | 0.5964 | 0.55 | 2.11 (2.13) | 0.32 | 1.98 (1.86) | 0.0091176 |
| 1523.74 | 40.66 | 0.7309 | 0.87 | 4.18 (4.21) | 0.89 | 3.83 (3.86) | 0.0052433 |
| 1523.84 | 29.75 | 0.5743 | 0.68 | 3.32 (3.39) | 0.53 | 3.18 (3.25) | 0.0003408 |
| 1525.48 | 37.17 | 0.6245 | 0.98 | 3.30 (3.32) | 0.94 | 3.19 (3.19) | 0.0199067 |
| 1526.69 | 23.92 | 0.6486 | 0.72 | 2.18 (2.18) | 0.49 | 2.01 (2.01) | 3.234E−05 |
| 1549.7 | 39.49 | 0.6466 | 0.7 | 2.33 (2.34) | 0.44 | 2.33 (2.34) | 0.0139286 |
| 1551.66 | 22.29 | 0.6124 | 0.64 | 2.31 (2.34) | 0.41 | 2.14 (2.18) | 7.338E−07 |
| 1552.5 | 37.22 | 0.7028 | 0.98 | 3.31 (3.32) | 0.91 | 3.09 (3.11) | 3.707E−05 |
| 1608.68 | 22.35 | 0.6205 | 0.68 | 2.31 (2.32) | 0.49 | 2.11 (2.15) | 2.061E−05 |
| 1608.73 | 30.93 | 0.6084 | 0.81 | 2.70 (2.66) | 0.64 | 2.45 (2.39) | 0.0009389 |
| 1612.76 | 23.38 | 0.6245 | 0.58 | 2.23 (2.24) | 0.31 | 2.06 (2.08) | 0.000304 |
| 1624.55 | 37.73 | 0.6586 | 0.97 | 3.05 (3.06) | 0.93 | 2.91 (2.90) | 0.0004413 |
| 1638.73 | 20.23 | 0.6245 | 0.63 | 2.68 (2.71) | 0.74 | 2.94 (2.94) | 0.0178203 |
| 1640.58 | 23.24 | 0.6305 | 0.91 | 3.60 (3.62) | 0.85 | 3.39 (3.44) | 0.0436627 |
| 1640.68 | 28.04 | 0.6747 | 0.33 | 1.85 (1.87) | 0.52 | 2.14 (2.17) | 0.0019075 |
| 1654.78 | 23.13 | 0.7149 | 0.25 | 2.08 (2.08) | 0.59 | 2.52 (2.52) | 1.37E−10 |
| 1664.75 | 29.81 | 0.6325 | 0.97 | 2.84 (2.86) | 0.87 | 2.77 (2.82) | 0.0003779 |
| 1669.69 | 21.47 | 0.7149 | 0.58 | 2.24 (2.24) | 0.75 | 2.71 (2.70) | 3.742E−06 |
| 1692.8 | 30.89 | 0.753 | 0.96 | 3.00 (3.04) | 0.67 | 2.65 (2.69) | 1.43E−13 |
| 1697.74 | 30.88 | 0.5863 | 0.97 | 3.03 (3.03) | 0.94 | 2.98 (3.00) | 0.0011862 |
| 1698.57 | 37.73 | 0.5763 | 0.61 | 2.79 (2.91) | 0.39 | 2.41 (2.41) | 0.0036709 |
| 1716.77 | 28 | 0.7149 | 0.89 | 2.72 (2.76) | 0.6 | 2.43 (2.52) | 3.30E−12 |
| 1725.59 | 38.32 | 0.6205 | 0.98 | 3.24 (3.25) | 0.9 | 3.09 (3.11) | 9.115E−05 |
| 1732.77 | 28.17 | 0.7329 | 0.92 | 3.44 (3.49) | 0.68 | 3.27 (3.32) | 2.937E−08 |
| 1749.81 | 30.61 | 0.6024 | 0.8 | 2.55 (2.53) | 0.64 | 2.35 (2.38) | 5.129E−06 |
| 1750.78 | 23.83 | 0.749 | 0.95 | 2.98 (3.06) | 0.71 | 2.60 (2.64) | 5.45E−12 |
| 1764.68 | 19.91 | 0.6024 | 0.57 | 2.53 (2.55) | 0.3 | 2.33 (2.33) | 8.56E−05 |
| 1765.81 | 31 | 0.6707 | 0.95 | 3.23 (3.26) | 0.82 | 3.04 (3.10) | 2.384E−05 |
| 1769.71 | 28.14 | 0.6526 | 0.59 | 2.14 (2.17) | 0.73 | 2.43 (2.49) | 0.0367372 |
| 1793.88 | 32.37 | 0.6506 | 0.58 | 2.14 (2.14) | 0.84 | 2.37 (2.43) | 1.598E−06 |
| 1798.72 | 36.95 | 0.6446 | 0.62 | 2.26 (2.35) | 0.27 | 2.02 (2.03) | 5.116E−07 |
| 1809.88 | 32.3 | 0.7369 | 0.19 | 1.85 (1.87) | 0.6 | 2.19 (2.22) | 7.12E−17 |
| 1817.69 | 20.23 | 0.6185 | 0.95 | 3.35 (3.43) | 0.81 | 3.20 (3.24) | 7.771E−06 |
| 1818.83 | 30.95 | 0.7731 | 0.69 | 2.30 (2.33) | 0.82 | 3.02 (3.20) | 4.883E−06 |
| 1822.73 | 30.87 | 0.6787 | 0.74 | 2.48 (2.51) | 0.41 | 2.50 (2.57) | 4.31E−09 |
| 1823.99 | 24.4 | 0.5341 | 0.62 | 2.69 (2.70) | 0.51 | 2.54 (2.52) | 0.0282935 |
| 1835.71 | 19.91 | 0.6084 | 0.77 | 2.87 (2.94) | 0.55 | 2.67 (2.68) | 2.228E−05 |
| 1837.8 | 30.56 | 0.6667 | 0.57 | 2.44 (2.46) | 0.16 | 2.33 (2.37) | 2.44E−15 |
| 1840.84 | 41.18 | 0.6205 | 0.62 | 2.38 (2.42) | 0.35 | 2.15 (2.32) | 2.254E−07 |
| 1847.89 | 43.67 | 0.6205 | 0.49 | 2.58 (2.66) | 0.14 | 2.30 (2.35) | 2.45E−12 |
| 1860.83 | 21.4 | 0.7631 | 0.89 | 2.87 (2.93) | 0.44 | 2.69 (2.77) | 1.06E−21 |
| 1880.9 | 43.91 | 0.6145 | 0.67 | 2.97 (3.09) | 0.45 | 2.60 (2.72) | 2.75E−10 |
| 1885.65 | 38.82 | 0.6546 | 0.75 | 2.14 (2.20) | 0.53 | 2.08 (2.10) | 5.025E−05 |
| 1892.86 | 24.33 | 0.6707 | 0.25 | 2.21 (2.18) | 0.53 | 2.32 (2.28) | 4.178E−07 |
| 1915.91 | 31.3 | 0.5924 | 0.52 | 2.07 (2.10) | 0.26 | 1.93 (1.97) | 1.28E−11 |
| 1916.77 | 20.32 | 0.6365 | 0.93 | 3.15 (3.23) | 0.78 | 3.01 (3.07) | 7.495E−05 |
| 1934.79 | 19.94 | 0.6185 | 0.72 | 2.75 (2.83) | 0.52 | 2.55 (2.62) | 0.0001583 |
| 1942.84 | 30.96 | 0.6325 | 0.68 | 2.07 (2.07) | 0.42 | 1.94 (1.95) | 7.525E−09 |
| 1945 | 33.71 | 0.5703 | 0.65 | 2.25 (2.31) | 0.87 | 2.32 (2.37) | 0.0050419 |
| 1962.88 | 31.81 | 0.6546 | 0.34 | 2.36 (2.43) | 0.64 | 2.34 (2.44) | 0.0004143 |
| 1963.88 | 31.74 | 0.6526 | 0.64 | 2.31 (2.33) | 0.32 | 2.29 (2.28) | 3.665E−06 |
| 1966.79 | 20.98 | 0.6205 | 0.83 | 2.84 (2.92) | 0.63 | 2.66 (2.68) | 3.313E−05 |
| 2034.99 | 40.19 | 0.5803 | 0.49 | 2.11 (2.20) | 0.23 | 1.89 (1.86) | 6.53E−11 |
| 2039.13 | 21.78 | 0.6185 | 0.74 | 2.97 (3.02) | 0.57 | 2.76 (2.96) | 6.134E−05 |
| 2055.94 | 25.44 | 0.6606 | 0.98 | 2.91 (2.96) | 0.9 | 2.85 (2.91) | 0.0340338 |
| 2058.94 | 23.15 | 0.6807 | 0.83 | 2.51 (2.57) | 0.49 | 2.30 (2.38) | 9.04E−12 |
| 2067.82 | 20.62 | 0.6365 | 0.92 | 3.08 (3.17) | 0.75 | 2.90 (2.98) | 1.957E−06 |
| 2070.92 | 25.4 | 0.747 | 0.96 | 2.99 (3.04) | 0.84 | 2.62 (2.67) | 1.033E−09 |
| 2076.95 | 21.78 | 0.6546 | 0.71 | 2.92 (2.98) | 0.43 | 2.44 (2.41) | 3.20E−12 |
| 2080.94 | 20.2 | 0.6747 | 0.81 | 2.84 (2.90) | 0.59 | 2.58 (2.62) | 0.0002311 |
| 2128.98 | 26.97 | 0.7028 | 0.69 | 2.14 (2.16) | 0.29 | 1.81 (1.84) | 1.95E−16 |
| 2132.91 | 25.83 | 0.6667 | 0.57 | 2.31 (2.33) | 0.69 | 2.69 (2.72) | 0.0073888 |
| 2137.94 | 21.79 | 0.6426 | 0.95 | 2.84 (2.87) | 0.82 | 2.74 (2.77) | 0.027956 |
| 2156.97 | 22.22 | 0.753 | 0.96 | 3.11 (3.15) | 0.71 | 2.72 (2.80) | 2.91E−14 |
| 2168.97 | 32.91 | 0.5361 | 0.71 | 2.74 (2.81) | 0.91 | 2.82 (2.86) | 0.0011953 |
| 2189 | 26.89 | 0.6586 | 0.93 | 2.95 (2.98) | 0.8 | 2.99 (3.00) | 0.014082 |
| 2191.99 | 22.39 | 0.7189 | 0.82 | 2.70 (2.73) | 0.51 | 2.24 (2.25) | 1.43E−14 |
| 2194.97 | 20.17 | 0.6365 | 0.58 | 2.52 (2.56) | 0.24 | 2.26 (2.55) | 4.695E−09 |
| 2216.03 | 33.83 | 0.7269 | 0.95 | 2.65 (2.69) | 0.87 | 2.24 (2.22) | 1.569E−09 |
| 2226.99 | 26.28 | 0.747 | 0.91 | 4.22 (4.21) | 0.63 | 3.90 (3.97) | 4.09E−11 |
| 2235.04 | 34.17 | 0.6888 | 0.88 | 2.89 (2.93) | 0.71 | 2.49 (2.49) | 3.61E−11 |
| 2248.99 | 25.99 | 0.5783 | 0.61 | 4.22 (4.23) | 0.88 | 4.20 (4.23) | 4.206E−05 |
| 2249.04 | 20.53 | 0.6145 | 0.68 | 2.59 (2.65) | 0.42 | 2.36 (2.36) | 4.476E−07 |
| 2257.87 | 35.93 | 0.6446 | 0.64 | 2.93 (2.93) | 0.32 | 2.41 (2.52) | 6.77E−14 |
| 2276.02 | 27.23 | 0.757 | 0.85 | 3.60 (3.68) | 0.41 | 3.34 (3.46) | 8.24E−17 |
| 2277.01 | 27.23 | 0.7048 | 0.29 | 2.86 (3.23) | 0.64 | 3.08 (3.20) | 3.207E−05 |
| 2280.94 | 36.22 | 0.5984 | 0.54 | 2.41 (2.45) | 0.24 | 2.07 (2.07) | 9.57E−10 |
| 2289.04 | 35.59 | 0.6044 | 0.56 | 2.21 (2.26) | 0.3 | 1.85 (1.86) | 6.98E−10 |
| 2292.02 | 27.28 | 0.747 | 1 | 3.68 (3.71) | 0.98 | 3.32 (3.39) | 1.419E−09 |
| 2308.02 | 27.34 | 0.6867 | 0.74 | 2.36 (2.43) | 0.37 | 2.02 (2.05) | 1.92E−11 |
| 2319.07 | 33.82 | 0.6024 | 0.51 | 2.93 (2.95) | 0.53 | 2.87 (2.91) | 0.0051152 |
| 2323.04 | 22.36 | 0.6486 | 0.81 | 2.52 (2.54) | 0.6 | 2.44 (2.49) | 0.0001244 |
| 2339 | 34.01 | 0.7631 | 0.94 | 2.91 (2.94) | 0.76 | 2.42 (2.50) | 1.23E−11 |
| 2367.06 | 27.63 | 0.7169 | 0.18 | 1.76 (1.75) | 0.5 | 2.07 (2.10) | 0.0105485 |
| 2377.1 | 20.8 | 0.7229 | 0.93 | 3.22 (3.32) | 0.78 | 2.83 (2.87) | 3.352E−10 |
| 2385.05 | 33.95 | 0.6426 | 0.93 | 2.88 (2.95) | 0.85 | 2.75 (2.84) | 0.0005018 |
| 2421 | 34.86 | 0.6606 | 0.58 | 1.96 (1.99) | 0.19 | 1.67 (1.77) | 9.64E−16 |
| 2446.09 | 28.37 | 0.6466 | 0.75 | 2.36 (2.39) | 0.54 | 2.14 (2.18) | 2.941E−06 |
| 2471.16 | 34.77 | 0.6767 | 0.93 | 2.65 (2.71) | 0.81 | 2.36 (2.43) | 2.143E−06 |
| 2483.12 | 27.57 | 0.7349 | 0.91 | 2.72 (2.80) | 0.71 | 2.34 (2.41) | 3.44E−13 |
| 2485.13 | 34.41 | 0.5924 | 0.61 | 2.22 (2.06) | 0.35 | 1.78 (1.80) | 2.10E−12 |
| 2525.2 | 27.74 | 0.6185 | 0.77 | 2.92 (3.00) | 0.65 | 2.70 (2.80) | 0.0031028 |
| 2544.13 | 28.26 | 0.5843 | 0.62 | 2.19 (2.25) | 0.49 | 2.22 (2.25) | 0.0162953 |
| 2559.18 | 19.41 | 0.6627 | 0.41 | 2.58 (2.63) | 0.58 | 3.04 (3.02) | 0.0386686 |
| 2570.19 | 42.56 | 0.5924 | 0.88 | 3.59 (3.68) | 0.82 | 3.44 (3.48) | 0.0252135 |
| 2583.15 | 23.68 | 0.6285 | 0.61 | 2.29 (2.29) | 0.34 | 2.27 (2.32) | 0.0001382 |
| 2584.23 | 35.18 | 0.6767 | 0.92 | 2.95 (3.00) | 0.86 | 2.64 (2.72) | 1.116E−07 |
| 2587.2 | 21.1 | 0.6667 | 0.64 | 2.72 (2.79) | 0.28 | 2.48 (2.53) | 2.24E−16 |
| 2596.23 | 34.9 | 0.6084 | 0.53 | 1.89 (1.92) | 0.21 | 1.62 (1.69) | 4.67E−11 |
| 2612.21 | 34.9 | 0.6225 | 0.73 | 2.74 (2.76) | 0.68 | 2.43 (2.50) | 0.0471086 |
| 2639.29 | 21.42 | 0.6265 | 0.73 | 2.54 (2.60) | 0.56 | 2.51 (2.53) | 7.298E−09 |
| 2644.22 | 21.15 | 0.6325 | 0.64 | 2.62 (2.62) | 0.35 | 2.32 (2.33) | 1.59E−12 |
| 2654.19 | 23.92 | 0.6345 | 0.89 | 2.39 (2.42) | 0.76 | 2.33 (2.40) | 0.0103611 |
| 2668.25 | 41.97 | 0.6406 | 0.74 | 2.57 (2.66) | 0.53 | 2.42 (2.54) | 8.044E−07 |
| 2748.79 | 36.38 | 0.6064 | 0.51 | 1.86 (1.88) | 0.22 | 1.72 (1.72) | 0.0003881 |
| 2751.34 | 29.23 | 0.5783 | 0.5 | 2.51 (2.58) | 0.27 | 2.30 (2.42) | 6.60E−13 |
| 2756.27 | 35.24 | 0.6426 | 0.77 | 2.36 (2.37) | 0.6 | 2.07 (2.08) | 0.000661 |
| 2767.32 | 21.67 | 0.6466 | 0.71 | 2.58 (2.64) | 0.47 | 2.39 (2.43) | 1.54E−16 |
| 2802.82 | 36.34 | 0.6365 | 0.5 | 2.17 (2.16) | 0.11 | 1.78 (1.83) | 3.46E−24 |
| 2839.35 | 24.2 | 0.5763 | 0.56 | 2.96 (3.10) | 0.43 | 2.49 (2.74) | 0.000138 |
| 2907.35 | 35.96 | 0.6044 | 0.78 | 2.38 (2.42) | 0.62 | 2.16 (2.17) | 0.0275969 |
| 2912.17 | 25.56 | 0.7189 | 0.98 | 2.97 (3.00) | 0.89 | 2.69 (2.76) | 2.093E−07 |
| 2926.3 | 22.22 | 0.6024 | 0.65 | 2.63 (2.64) | 0.45 | 2.51 (2.55) | 0.0054093 |
| 2939.15 | 33.77 | 0.6004 | 0.63 | 2.33 (2.38) | 0.44 | 2.05 (2.10) | 5.882E−06 |
| 2946.21 | 34.98 | 0.6165 | 0.42 | 1.89 (1.84) | 0.61 | 2.05 (2.14) | 2.307E−05 |
| 2973.45 | 24.37 | 0.6145 | 0.75 | 2.68 (2.71) | 0.59 | 2.59 (2.57) | 3.743E−07 |
| 3001.43 | 35.4 | 0.6446 | 0.83 | 3.87 (3.95) | 0.77 | 3.61 (3.75) | 0.0363329 |
| 3002.24 | 23.8 | 0.6466 | 0.59 | 2.03 (2.04) | 0.26 | 1.83 (1.88) | 7.23E−10 |
| 3011.39 | 29.75 | 0.6867 | 0.95 | 3.31 (3.36) | 0.91 | 3.14 (3.17) | 0.0003048 |
| 3013.29 | 22.3 | 0.7229 | 0.86 | 3.75 (3.82) | 0.69 | 3.16 (3.35) | 8.80E−11 |
| 3035.19 | 42.02 | 0.5643 | 0.68 | 2.62 (2.70) | 0.55 | 2.36 (2.43) | 0.0066459 |
| 3064.32 | 20.57 | 0.6345 | 0.6 | 2.70 (2.74) | 0.31 | 2.50 (2.53) | 7.237E−07 |
| 3081.42 | 29.83 | 0.5984 | 0.57 | 2.49 (2.59) | 0.31 | 2.31 (2.38) | 2.315E−06 |
| 3091.44 | 28.4 | 0.7048 | 0.8 | 2.75 (2.79) | 0.66 | 2.39 (2.45) | 4.189E−08 |
| 3108.45 | 31.28 | 0.6566 | 0.95 | 2.73 (2.78) | 0.86 | 2.60 (2.61) | 0.0016437 |
| 3139.49 | 29.48 | 0.5743 | 0.7 | 2.94 (2.99) | 0.59 | 2.83 (2.81) | 0.0458493 |
| 3149.46 | 31.25 | 0.6667 | 0.89 | 2.79 (2.85) | 0.78 | 2.59 (2.60) | 7.125E−05 |
| 3165.46 | 31.32 | 0.6526 | 0.85 | 2.54 (2.58) | 0.74 | 2.32 (2.32) | 0.005123 |
| 3178.43 | 30.3 | 0.7309 | 0.4 | 2.06 (2.10) | 0.65 | 2.58 (2.62) | 1.023E−08 |
| 3193.38 | 22.64 | 0.6647 | 0.7 | 3.17 (3.23) | 0.38 | 2.81 (2.89) | 3.766E−08 |
| 3209.41 | 22.67 | 0.7129 | 0.96 | 3.77 (3.79) | 0.86 | 3.55 (3.63) | 1.663E−05 |
| 3256.53 | 33.03 | 0.6988 | 0.7 | 2.78 (2.80) | 0.3 | 2.63 (2.61) | 1.121E−06 |
| 3281.43 | 36.09 | 0.6406 | 0.96 | 3.17 (3.23) | 0.88 | 3.08 (3.13) | 0.0046604 |
| 3292.54 | 39.42 | 0.6586 | 0.9 | 3.62 (3.69) | 0.74 | 3.43 (3.49) | 1.357E−09 |
| 3318.55 | 30.99 | 0.6104 | 0.71 | 2.27 (2.30) | 0.49 | 2.09 (2.10) | 9.729E−05 |
| 3333.72 | 23.83 | 0.747 | 0.18 | 2.20 (2.26) | 0.55 | 2.80 (2.89) | 5.19E−10 |
| 3334.54 | 31.02 | 0.5482 | 0.48 | 2.47 (2.56) | 0.33 | 2.17 (2.24) | 4.246E−05 |
| 3337.45 | 22.81 | 0.6627 | 0.68 | 2.89 (2.94) | 0.36 | 2.47 (2.54) | 4.37E−18 |
| 3350.55 | 31.02 | 0.5924 | 0.58 | 2.24 (2.25) | 0.44 | 1.95 (2.00) | 0.0044159 |
| 3359.58 | 31.9 | 0.6968 | 0.94 | 3.23 (3.28) | 0.91 | 2.93 (2.96) | 0.0059278 |
| 3363.54 | 30.22 | 0.747 | 0.39 | 2.07 (2.08) | 0.65 | 2.51 (2.50) | 4.15E−08 |
| 3385.55 | 25.49 | 0.6486 | 0.78 | 3.53 (3.59) | 0.56 | 3.25 (3.35) | 1.93E−13 |
| 3401.6 | 25.47 | 0.6325 | 0.85 | 2.98 (3.06) | 0.76 | 2.82 (2.87) | 4.667E−08 |
| 3405.48 | 25.97 | 0.5482 | 0.53 | 3.41 (3.50) | 0.42 | 3.44 (3.51) | 0.0079805 |
| 3426.31 | 27.7 | 0.6225 | 0.44 | 1.94 (2.00) | 0.6 | 2.11 (2.15) | 0.002223 |
| 3575.75 | 32.36 | 0.6707 | 0.31 | 1.94 (1.94) | 0.58 | 2.20 (2.17) | 1.274E−05 |
| 3657.67 | 40.71 | 0.6124 | 0.7 | 2.93 (3.00) | 0.55 | 2.67 (2.71) | 2.29E−06 |
| 3696.76 | 26.94 | 0.5904 | 0.6 | 2.37 (2.43) | 0.38 | 2.25 (2.27) | 2.447E−06 |
| 3718.72 | 32.48 | 0.6988 | 0.92 | 3.05 (3.11) | 0.81 | 2.78 (2.82) | 1.69E−06 |
| 3734.72 | 32.5 | 0.6586 | 0.88 | 2.83 (2.91) | 0.83 | 2.56 (2.57) | 1.702E−05 |
| 3831.81 | 28.48 | 0.6205 | 0.73 | 2.90 (3.00) | 0.55 | 2.74 (2.78) | 0.0031118 |
| 3870.81 | 33.49 | 0.5643 | 0.57 | 2.20 (2.23) | 0.37 | 1.97 (2.04) | 0.0003836 |
| 3871.79 | 27.57 | 0.5823 | 0.6 | 2.48 (2.55) | 0.41 | 2.40 (2.48) | 0.0280454 |
| 3943.83 | 33.63 | 0.6165 | 0.78 | 2.39 (2.44) | 0.72 | 2.13 (2.14) | 0.0186637 |
| 4002.62 | 20.66 | 0.5502 | 0.71 | 2.95 (3.07) | 0.63 | 2.83 (2.87) | 0.0051078 |
| 4047.92 | 25.45 | 0.6165 | 0.42 | 2.18 (2.21) | 0.59 | 2.33 (2.30) | 0.0148071 |
| 4078.81 | 33.14 | 0.7289 | 0.42 | 2.00 (2.00) | 0.66 | 2.41 (2.38) | 1.67E−05 |
| 4217.98 | 26.05 | 0.6205 | 0.68 | 3.49 (3.59) | 0.51 | 3.31 (3.37) | 1.7E−05 |
| 4251.98 | 28.76 | 0.6466 | 0.78 | 3.00 (3.05) | 0.66 | 2.74 (2.80) | 1.238E−06 |
| 4289.93 | 28.78 | 0.6546 | 0.83 | 3.67 (3.70) | 0.63 | 3.54 (3.60) | 0.0018768 |
| 4368.9 | 20.21 | 0.5643 | 0.68 | 3.10 (3.13) | 0.6 | 3.01 (3.05) | 0.0436271 |
| 4436.08 | 26.32 | 0.5843 | 0.58 | 3.29 (3.31) | 0.42 | 3.12 (3.13) | 0.0008656 |
| 4467.96 | 29.12 | 0.5361 | 0.53 | 2.62 (2.71) | 0.43 | 2.43 (2.52) | 0.0015007 |
| 4630 | 29.38 | 0.5984 | 0.6 | 2.73 (2.78) | 0.39 | 2.58 (2.66) | 3.49E−06 |
| 4863.16 | 26.74 | 0.5884 | 0.67 | 2.72 (2.88) | 0.57 | 2.69 (2.79) | 0.0004877 |
| 8917.25 | 22.55 | 0.6245 | 0.37 | 2.43 (2.46) | 0.55 | 2.55 (2.47) | 0.0004797 |

TABLE 3

| | | | | | |
|---|---|---|---|---|---|
| | | | | Protein | Swissprot |
| Mass | CE T | Sequence | Protein name | coverage | Name |

| | | | | | |
|---|---|---|---|---|---|
| 858.39 | 23.24 | | | | |
| 884.32 | 24.85 | | | | |
| 911.43 | 25.88 | | | | |
| 981.59 | 24.8 | VLNLGPITR | Uromodulin | 598-606 | UROM_HUMAN |
| 984.46 | 24.92 | | | | |
| 1013.37 | 25.17 | | | | |
| 1018.46 | 24.54 | | | | |
| 1040.47 | 25.05 | SPhGPDGKTGPPh | Collagen alpha-1 (I) chain | 546-556 | CO1A1 HUMAN |
| 1050.48 | 26.92 | MGPRGPPhGPPhG | Collagen alpha-1 (I) chain | 217-227 | CO1A1_HUMAN |
| 1058.48 | 24.89 | | | | |
| 1070.49 | 36.49 | | | | |
| 1071.49 | 21.43 | | | | |
| 1080.48 | 27.77 | | | | |
| 1080.5 | 25.69 | | | | |
| 1096.48 | 26 08 | APhGDRGEPhGPPh | Collagen alpha-1 (I) chain | 798-808 | CO1A1_HUMAN |
| 1100.5 | 37.04 | | | | |
| 1114.49 | 25.55 | | | | |
| 1128.49 | 25.65 | | | | |
| 1141.52 | 24.51 | _ | | | |
| 1141.54 | 37.33 | GPPhGPhPGPPGPPS | Collagen alpha-1 (I) chain | 1181-1193 | CO1A1_HUMAN |
| 1143.52 | 36.97 | | | | |
| 1153.31 | 35.61 | | | | |
| 1157.54 | 37.44 | GPPGPhPhGPhPGPPS | Collagen alpha-1 (I) chain | 1181-1193 | COIA1_HUMAN |
| 1162.54 | 20.11 | | | | |
| 1173.53 | 37.49 | | | | |
| 1180.52 | 35.7 | | | | |
| 1182.55 | 28.27 | | | | |
| 1186.53 | 22.39 | DDGEAGKPhGRPhG | Collagen alpha-1 (I) chain | 231-242 | CO1A1_HUMAN |
| 1191.52 | 36.18 | | | | |
| 1210.39 | 36.48 | | | | |
| 1211.54 | 25.82 | | | | |
| 1216.54 | 24.24 | | | | |
| 1217.53 | 35.78 | | | | |
| 1260.56 | 21.83 | | | | |
| 1262.46 | 38.23 | | | | |
| 1263.54 | 22.73 | | | | |
| 1265.59 | 27.09 | SPhGPDGKTGPPhGPA | Collagen alpha-1 (I) chain | 546-559 | CO1A1_HUMAN |
| 1270.55 | 29.38 | | | | |
| 1281.58 | 27.09 | | | | |
| 1297.58 | 27.36 | SPhGSPhGPDGKTGPPh | 543-556 | CO1A1_HUMAN | |
| 1299.58 | 22.38 | | | | |
| 1312.55 | 29.77 | | | | |
| 1312.62 | 22.45 | | | | |
| 1324.59 | 28.7 | | | | |
| 1326.55 | 29.2 | SPhGGPhGSDGKPhGPPhG | Collagen alpha-1 (III) chain | 541-555 | CO3A1_HUMAN |
| 1337.62. | 38.2 | | | | |
| 1351.64 | 38.76 | | | | |
| 1353.66 | 25.63 | | | | |
| 1367.64 | 38.88 | | | | |
| 1378.61 | 28.82 | APhGEDGRPhGPPhGPQ | Collagen alpha-1 (II) chain | 511-524 | CO2A1_HUMAN |
| 1383.64 | 38.94 | | | | |
| 1396.62 | 26.67 | | | | |
| 1405.64 | 20.14 | DGPPhGRDGQPhGHKG | Collagen alpha-2 (I) chain | 933-946 | CO1A2_HUMAN |
| 1407.65 | 37.23 | | | | |
| 1408.66 | 39.13 | | | | |
| 1422.68 | 28.14 | | | | |
| 1424.66 | 39.3 | GLPGPPhGPPhGSFLSN | Collagen alpha-1 (XVII) chain | 885-899 | COHA1_HUMAN |
| 1438.66 | 30.2 | GLPhGTGGPPhGENGKPhG | Collagen alpha-1 (III) chain | 642-657 | CO3A1_HUMAN |
| 1438.67 | 27.88 | | | | |
| 1439.66 | 29.82 | TIDEKGTEAAGAMF | Alpha-1-antitrypsin | 328-341 | A1AT_HUMAN |
| 1440.66 | 39.28 | | | | |
| 1442.63 | 27.63 | | | | |
| 1458.63 | 27.94 | SPhGENGAPhGQMoxGPRG | Collagen alpha-1 (I) chain | 291-305 | CO1A1_HUMAN |
| 1470.68 | 21.08 | | | | |
| 1482.67 | 22.47 | | | | |
| 1485.67 | 23.77 | DGQPhGAKGEPhGDAGAK | Collagen alpha-1 (I) chain | 820-835 | CO1A1-HUMAN |
| 1486.68 | 21.15 | | | | |
| 1491.74 | 39.83 | VGPPhGPhPGPPGPPGPPS | Collagen alpha-1 (I) chain | 1174-1190 | CO1A1_HUMAN |
| 1494.66 | 30.4 | | | | |
| 1496.68 | 30.38 | | | | |
| 1507.74 | 40.02 | | | | |
| 1508.68 | 29.33 | GSPhGSPhGPDGKTGPPGPh | Collagen alpha-1 (I) chain | 542-558 | CO1A1-HUMAN |
| 1510.68 | 20.17 | | | | |
| 1513.44 | 36.79 | | | | |
| 1521.69 | 30.53 | GDSDDDEPPPLPRL | Membrane associated | 54-67 | PGRC1_HUMAN |
| | | | progesterone receptor | | |
| | | | component 1 | | |
| 1523.74 | 40.66 | GDPGPPGPhPGPhPGPhPAI | Collagen alpha-1 (XV) chain | 1093-1109 | COFA1_HUMAN |
| 1523.84 | 29.75 | VIDQSRVLNLGPIT | Uromodulin | 592-605 | UROM_HUMAN |
| 1525.48 | 37.17 | | | | |
| 1526.69 | 23.92 | | | | |
| 1549.7 | 39.49 | | | | |
| 1551.66 | 22.29 | | | | |
| 1552.5 | 37.22 | | | | |
| 1608.68 | 22.35 | | | | |
| 1608.73 | 30.93 | SGDSDDDEPPPLPRL | Membrane associated | 53-67 | PGRC1_HUMAN |
| | | | progesterone receptor | | |
| | | | component 1 | | |
| 1612.76 | 23.38 | APGSKGDTGAKGEPGPVG | Collagen alpha-1 (I) chain | 438-455 | CO1A1-HUMAN |
| 1638.73 | 20.23 | AGSEADHEGTHSTKRG | Fibrinogen alpha chain | 607-622 | FIBA_HUMAN |
| 1640.58 | 23.24 | | | | |
| 1640.68 | 28.04 | | | | |
| 1654.78 | 28.13 | | | | |
| 1664.75 | 29.81 | | | | |
| 1669.69 | 21.47 | DEAGSEADHEGTHSTK | Fibrinogen alpha chain | 605-620 | FIBA_HUMAN |
| 1692.8 | 30.89 | PPhGEAGKPhGEQGVPGDLG | Collagen alpha-1 (I) chain | 651-668 | CO1A1_HUMAN |
| 1697.74 | 30.88 | NGAPGNDGAKGDAGAPGAPG | Collagen alpha-1 (I) chain | 700-719 | CO1A1_HUMAN |
| 1698.57 | 37.73 | | | | |
| 1716.77 | 28 | | | | |
| 1725.59 | 38.32 | | | | |
| 1732.77 | 28.17 | | | | |
| 1749.81 | 30.61 | GPPhGEAGKPhGEQGVPGDLG | Collagen alpha-1 (I) chain | 650-668 | CO1A1_HUMAN |
| 1750.78 | 23.83 | GPPhGPPhGKNGDDGEAGKPhG | Collagen alpha-1 (I) chain | 221-239 | CO1A1_HUMAN |
| 1764.68 | 19.91 | | | | |
| 1765.81 | 31 | GPPhGEAGKPhGEQGVPhGDLG | Collagen alpha-1 (I) chain | 650-668 | CO1A1_HUMAN |
| 1769.71 | 28.14 | | | | |
| 1793.88 | 32.37 | EEAPSLRPAPPPISGGGY | .Fibrinogen beta chain | 54-71 | FIBS_HUMAN |
| 1798.72 | 36.95 | | | | |
| 1809.88 | 32.3 | | | | |
| 1817.69 | 20.23 | | | | |
| 1818.83 | 30.95 | | | | |
| 1822.73 | 30.87 | | | | |
| 1823.99 | 24.4 | GSVIDQSRVLNLGPITR | Uromodulin | 590-606 | UROM_HUMAN |
| 1835.71 | 19.91 | | | | |
| 1837.8 | 30.56 | | | | |
| 1840.84 | 41.18 | | | | |
| 1847.89 | 43.67 | | | | |
| 1860.83 | 21.4 | EGSPhGRDGSPhGAKGDRGET | Collagen alpha-1 (I) chain | 1021-1039 | CO1A1_HUMAN |
| 1880.9 | 43.91 | | | | |
| 1885.65 | 38.82 | | | | |
| 1892.86 | 24.33 | | | | |
| 1915.91 | 31.3 | | | | |
| 1916.77 | 20.32 | | | | |
| 1934.79 | 19.94 | | | | |
| 1942.84 | 30.96 | | | | |
| 1945 | 33.71 | | | | |
| 1962.88 | 31.81 | | | | |
| 1963.88 | 31.74 | | | | |
| 1996.79 | 20.98 | | | | |
| 2034.99 | 40.19 | | | | |
| 2039.13 | 21.78 | SGSVIDQSRVLNLGPITRK | Uromoduolin | 589-607 | UROM_HUMAN |
| 2055.94 | 25.44 | | | | |
| 2058.94 | 23.15 | | | | |
| 2067.82 | 20.62 | | | | |
| 2070.92 | 25.4 | GNSGEPhGAPhGSKGDTGAKGETGPh | Collagen alpha-1 (I) chain | 431-453 | CO1A1_HUMAN |
| 2076.95 | 21.78 | | | | |
| 2080.94 | 20.2 | DAHKSEVAHRFKDLGEEN | Serum albumin | 25-42; N-term | ALBU-HUMAN |
| 2128.98 | 26.97 | DGKTGPhPGPAGQDGRPGPPhGPhPhG | Collagen alpha-1 (I) chain | 550-572 | CO1A1_HUMAN |
| 2132.91 | 25.83 | | | | |
| 2137.94 | 21.79 | NGEPhGGKGERGAPhGEKGEGGPhPG | Collagen alpha-1 (III) chain | 818-840 | CO3A1_HUMAN |
| 2156.97 | 22.22 | AEGSPhGRDGSPhGAKGDRGETGPA | Collagen alpha-1 (I) chain | 1020-1042 | CO1A1_HUMAN |
| 2168.97 | 32.91 | | | | |
| 2189 | 26.89 | ADGQPGAKGEPGDAGAKGDAGPPhGP | Collagen alpha-1 (I) chain | 819-843 | CO1A1_HUMAN |
| 2191.99 | 22.39 | | | | |
| 2194.97 | 20.17 | NDGPPhGRDGQPhGHKGETGYPhG | Collagen alpha-2 (I) chain | 932-952 | CO1A2_HUMAN |
| 2216.03 | 33.83 | | | | |
| 2226.99 | 26.28 | GNSGEPhGAPhGSKGDTGAKGEPhGPVG | Collagen alpha-1 (I) chain | 431-455 | CO1A1_HUMAN |
| 2235.04 | 34.17 | GRTGDAGPVGPPGPPhGPHPhGPhPGPPS | Collagen alpha-1 (I) chain | 1169-1193 | CO1A1_HUMAN |
| 2248.99 | 25.99 | | | | |
| 2249.04 | 20.53 | GKNGDDGEQGKPhGRPhGERGPhPGP | Collagen alpha-1 (I) chain | 227-249 | CO1A1_HUMAN |
| 2257.87 | 35.93 | | | | |
| 2276.02 | 27.23 | ADGQPGAKGEPhGKAGAKGDAGPPGPhA | Collagen alpha-1 (I) chain | 819-944 | CO1A1_HUMAN |
| 2277.01 | 27.23 | | | | |
| 2280.94 | 36.22 | | | | |
| 2289.04 | 33.59 | | | | |
| 2292.02 | 27.28 | ADGPQhGAKGEPhGDAGAKGDAGPPhGPA | Collagen alpha-1 (I) chain | 819-844 | CO1A1_HUMAN |
| 2308.02 | 27.34 | ADGQPhGAKGEPhGDAGAKGDAGPhPhGPA | Collagen alpha-1 (I) chain | 819-944 | CO1A1_HUMAN |
| 2319.07 | 33.82 | | | | |
| 2323.04 | 22.36 | GQNGEPhGGKGERGAPhGEKGEGGPhG | Collagen alpha-1 (III) chain | 816-840 | CO1A3_HUMAN |
| 2367.06 | 27.63 | | | | |
| 2377.1 | 20.8 | GKNGDDGEGKhPGRPhGERGPPhGPQ | Collagen alpha-1 (I) chain | 227-252 | CO1A1_HUMAN |
| 2385.05 | 33.95 | | | | |
| 2421 | 34.86 | | | | |
| 2446.09 | 28.37 | ADGQPhGAKGEPhGDAGADGDAGPhPGPAGP | Collagen alpha-1 (I) chain | 819-846 | CO1A1_HUMAN |
| 2471.16 | 34.77 | TGPIGPPhGPAGAPhGDKGESGPSGPAGPTG | Collagen alpha-1 (I) chain | 766-794 | CO1A1_HUMAN |
| 2483.12 | 27.57 | | | | |
| 2485.13 | 34.41 | | | | |
| 2525.2 | 27.74 | | | | |
| 2544.13 | 28.26 | | | | |
| 2559.18 | 19.41 | DEAGSEADHEFTHSTKRGHAKSRP | Fibrinogen alpha chain | 605-628 | FIBA_HUMAN |
| 2570.19 | 42.56 | | | | |
| 2583.15 | 23.68 | | | | |
| 2584.35 | 35.18 | | | | |
| 2587.2 | 21.1 | | | | |
| 2596.23 | 34.9 | | | | |
| 2612.21 | 34.9 | | | | |
| 2639.29 | 21.42 | | | | |
| 2644.22 | 21.15 | | | | |
| 2654.19 | 23.92 | ERGEAGIPhGVPhGAKGEDGKDGSPhGEPhGA | Collagen alpha-1 (III) chain | 448-475 | CO3A1_HUMAN |
| 2668.25 | 41.97 | | | | |
| 2748.79 | 36.38 | | | | |
| 2751.34 | 29.23 | | | | |
| 2756.27 | 35.24 | | | | |
| 2767.32 | 21.67 | KEGGKGPRGETGPAGRPhGEVGPhPGPPhGPAG | Collagen alpha-1 (I) chain | 906-935 | CO1A1_HUMAN |
| 2802.82 | 36.34 | | | | |
| 2839.35 | 24.2 | | | | |
| 2907.35 | 35.96 | | | | |
| 2912.17 | 25.56 | | | | |
| 2926.3 | 22.22 | ESGREGAPGAEGSPhGRDGSPhGAKGDRGETGP | Collagen alpha-1 (I) chain | 1011-1041 | CO1A1_HUMAN |
| 2939.15 | 33.77 | | | | |
| 2946.21 | 34.98 | | | | |
| 2973.45 | 24.37 | | | | |
| 3001.43 | 35.4 | | | | |
| 3002.24 | 23.8 | | | | |
| 3011.39 | 29.75 | LTGSPhGSPhGPhDGKTGPPGPAGQDGRPGPP | Collagen alpha-1 (I) chain | 537-569 | CO1A1_HUMAN |
| | | hGPhPhG | | | |
| 3013.29 | 22.3 | ESGREGAPhGAEGSPhGRDGSPhGAKGDRGET | Collagen alpha-1 (I) chain | 1011-1040 | CO1A1_HUMAN |
| | | GPA | | | |
| 3035.19 | 42.02 | | | | |
| 3064.32 | 20.57 | | | | |
| 3081.42 | 29.83 | | | | |
| 3091.44 | 28.4 | | | | |
| 3108.45 | 31.28 | ADGQPhGAKGEPhGDAGAKGDAGPhPGPAGPA | Collagen alpha-1 (I) chain | 819-854 | CO1A1_HUMAN |
| | | GPPGPhIG | | | |
| 3139.49 | 29.48 | | | | |
| 3149.46 | 31.25 | GADGQPGAKGEPhGDAGAKGDAGPPhGPAGPh | Collagen alpha-1 (I) chain | 818-854 | CO1A1_HUMAN |
| | | AGPPGPIG | | | |
| 3165.46 | 31.32 | | | | |
| 3178.43 | 30.3 | | | | |
| 3193.38 | 22.64 | PPhGESGREGAPGAEGSPhGRDGSPhGAKGDR | Collagen alpha-1 (I) chain | 1008-1041 | CO1A1_HUMAN |
| | | GETGP | | | |
| 3209.41 | 22.67 | PPhGESGREGAPhGAEGSPhGRDGSPhGAKGD | Collagen alpha-1 (I) chain | 1008-1041 | CO1A1_HUMAN |
| | | RGETGP | | | |
| 3256.53 | 33.03 | | | | |
| 3281.43 | 36.09 | | | | |
| 3292.54 | 39.42 | | | | |
| 3318.55 | 30.99 | | | | |
| 3333.72 | 23.83 | | | | |
| 3334.54 | 31.02 | | | | |
| 3337.45 | 22.81 | GPPhGESGREGAPhGAEGSPhGRDGSPhGAKG | Collagen alpha-1 (I) chain | 1007-1042 | CO1A1_HUMAN |
| | | DRGETGPA | | | |
| 3350.55 | 31.02 | | | | |
| 3359.58 | 31.9 | | | | |
| 3363.54 | 30.22 | | | | |
| 3385.55 | 25.49 | | | | |
| 3401.6 | 25.47 | | | | |
| 3405.48 | 25.97 | ARGNDGARGSDGQPGPPhGPhPhGTAGFPhGS | Collagen alpha-1 (III) chain | 319-355 | CO3A1_HUMAN |
| | | PhGAKGEVGP | | | |
| 3426.31 | 27.7 | | | | |
| 3575.75 | 32.36 | | | | |
| 3657.67 | 40.71 | | | | |
| 3696.76 | 26.94 | | | | |
| 3718.72 | 32.48 | | | | |
| 3734.72 | 32.5 | | | | |
| 3831.81 | 28.48 | | | | |
| 3870.81 | 33.49 | | | | |
| 3871.79 | 27.57 | | | | |
| 3943.83 | 33.63 | | | | |
| 4002.62 | 20.66 | | | | |
| 4047.92 | 25.45 | | | | |
| 4078.81 | 33.14 | | | | |
| 4217.98 | 26.05 | EEKAVADTRDQADGSRASVDSGSSEEQGGSSR | Polymeric-immunoglobulin | 607-648 | PIGR_HUMAN |
| | | ALVSTLVPGL | receptor | | |
| 4289.93 | 28.78 | ARGNDGARGSDGQPhGPhPhGPPGTAGFPGS- | Collagen alpha-1 (III) chain | CO3A1_HUMAN | |
| | | PhGAKGEVGPhAGSPhGSNGAPhG | | | |
| 43683.9 | 20.21 | | | | |
| 4436.08 | 26.32 | | | | |
| 4467.96 | 29.12 | | | | |
| 4630 | 29.38 | | | | |
| 4863.16 | 26.74 | | | | |
| 8917.25 | 22.55 | | | | |

1. A process for the diagnosis of diabetes mellitus comprising the step of determining the presence or absence or amplitude of at least three polypeptide markers in a urine sample, the polypeptide markers being selected from the markers characterized by the values for the molecular masses and migration times according to the following Table:

| No. | Mass (Da) | CE T (min) |
|---|---|---|
| 1 | 858.39 | 23.24 |
| 2 | 884.32 | 24.85 |
| 3 | 911.43 | 25.88 |
| 4 | 981.59 | 24.8 |
| 5 | 984.46 | 24.92 |
| 6 | 1013.37 | 25.17 |
| 7 | 1018.46 | 24.54 |
| 8 | 1040.47 | 25.05 |
| 9 | 1050.48 | 26.92 |
| 10 | 1058.48 | 24.89 |
| 11 | 1070.49 | 36.49 |
| 12 | 1071.49 | 21.43 |
| 13 | 1080.48 | 27.77 |
| 14 | 1080.5 | 25.69 |
| 15 | 1096.48 | 26.08 |
| 16 | 1100.5 | 37.04 |
| 17 | 1114.49 | 25.55 |
| 18 | 1128.49 | 25.65 |
| 19 | 1141.52 | 24.51 |
| 20 | 1141.54 | 37.33 |
| 21 | 1143.52 | 36.97 |
| 22 | 1153.31 | 35.61 |
| 23 | 1157.54 | 37.44 |
| 24 | 1162.54 | 20.11 |
| 25 | 1173.53 | 37.49 |
| 26 | 1180.52 | 35.7 |
| 27 | 1182.55 | 28.27 |
| 28 | 1186.53 | 22.39 |
| 29 | 1191.52 | 36.18 |
| 30 | 1210.39 | 36.48 |
| 31 | 1211.54 | 25.82 |
| 32 | 1216.54 | 24.24 |
| 33 | 1217.53 | 35.78 |
| 34 | 1260.56 | 21.83 |
| 35 | 1262.46 | 38.23 |
| 36 | 1263.54 | 22.73 |
| 37 | 1265.59 | 27.09 |
| 38 | 1270.55 | 29.38 |
| 39 | 1276.4 | 35.92 |
| 40 | 1281.58 | 27.09 |
| 41 | 1297.58 | 27.36 |
| 42 | 1299.58 | 22.38 |
| 43 | 1312.55 | 29.77 |
| 44 | 1312.62 | 22.45 |
| 45 | 1324.59 | 28.7 |
| 46 | 1326.55 | 29.2 |
| 47 | 1337.62 | 38.2 |
| 48 | 1351.64 | 38.76 |
| 49 | 1353.66 | 25.63 |
| 50 | 1367.64 | 38.88 |
| 51 | 1378.61 | 28.82 |
| 52 | 1383.64 | 38.94 |
| 53 | 1396.62 | 26.67 |
| 54 | 1405.64 | 20.14 |
| 55 | 1407.66 | 37.23 |
| 56 | 1408.66 | 39.13 |
| 57 | 1422.68 | 28.14 |
| 58 | 1424.66 | 39.3 |
| 59 | 1438.66 | 30.2 |
| 60 | 1438.67 | 27.88 |
| 61 | 1439.66 | 29.82 |
| 62 | 1440.66 | 39.28 |
| 63 | 1442.63 | 27.63 |
